(19) United States
(12) Patent Application Publication
(10) Pub. No.: US 2022/0096802 A1
(43) Pub. Date: Mar. 31, 2022

(54) GUIDE WIRE

(71) Applicant: ASAHI INTECC CO., LTD., Seto-shi (JP)

(72) Inventor: Kenta TSUGE, Seto-shi (JP)

(73) Assignee: ASAHI INTECC CO., LTD., Seto-shi (JP)

(21) Appl. No.: 17/546,062

(22) Filed: Dec. 9, 2021

Related U.S. Application Data
(63) Continuation of application No. PCT/JP2019/024804, filed on Jun. 21, 2019.

(51) Int. Cl. A61M 25/09

(52) U.S. Cl. CPC A61M 25/09; A61M 2025/09083

(57) ABSTRACT

A guide wire includes a core shaft having a first expanded diameter portion which increases in diameter toward the proximal end side and a first extended portion, a first coil body wound so as to cover at least a portion of the core shaft, a distal end fixing portion at which the distal end of the core shaft and the distal end of the first coil body are fixed to each other and a proximal end of the first coil body is not fixed to the first expanded diameter portion, but is fixed to the first extended portion.

### CROSS-REFERENCE TO RELATED APPLICATION

This application is a continuation application of International Application No. PCT/JP2019/024804, filed Jun. 21, 2019. The contents of this application are incorporated herein by reference in its entirety.

### TECHNICAL FIELD

The present disclosure relates to a guide wire.

### BACKGROUND

When treating a site such as a constricted part that has formed inside a blood vessel, a guide wire is inserted prior to a treatment device such as a catheter.

In order to enable the guide wire to proceed through complicated, curved blood vessels and reach the treatment site with certainty, the guide wire is required to have a flexible distal end portion, and torquability so that rotation operations performed by hand can be propagated with certainty to the distal end portion of the guide wire.

As an example of such a guide wire, a guide wire provided with a core wire having a tapered portion, and a coil wound to cover the core wire is known (see JPH7-227429, for example).

According to the guide wire described above, it is possible to obtain flexibility of the distal end portion due to the core wire having a reduced diameter and a high torquability due to the coil, and therefore, smooth insertion into a treatment site inside a blood vessel can be expected.

Although the conventional guide wire described above can be expected to provide an improvement in terms of flexibility and torquability, the proximal end portion of a U-shaped curved portion formed on the distal end portion of the guide wire may proceed beyond the tapered portion of the core wire to the proximal end side, or the core wire at this part may become plastically bent, which may hinder the smooth insertion into a blood vessel.

### SUMMARY

The present disclosure has been made based on the above circumstances. One or more embodiments are to provide a guide wire with which it is possible to prevent a proximal end portion of a U-shaped curved portion formed on a distal end portion of a guide wire from proceeding beyond an expanded diameter portion to the proximal end side, and prevent plastic deformation from occurring in the core shaft at the expanded diameter portion.

One or more embodiments provide a guide wire that includes a core shaft having a first expanded diameter portion which expands in diameter toward a proximal end side and a first extended portion having a cylindrical shape or a truncated cone shape that becomes thinner toward a distal end side, which is provided adjacent to the first expanded diameter portion so as to extend along a longitudinal axis direction, a first coil body wound so as to cover at least a portion of the core shaft, and a distal end fixing portion at which a distal end of the core shaft and a distal end of the first coil body are fixed to each other. A gradient of a proximal end of the first expanded diameter portion may be greater than a gradient of a distal end of the first expanded diameter portion. An average gradient between a distal end of the first expanded diameter portion and a proximal end of the first expanded diameter portion may be greater than a gradient of the first extended portion. A gradient of the first expanded diameter portion may increase continuously toward a proximal end side. A proximal end of the first coil body may not be fixed to the first expanded diameter portion, but may be fixed to the first extended portion.

### BRIEF DESCRIPTION OF THE DRAWINGS

FIG. 1A is a schematic side view showing an embodiment;

FIG. 1B is a schematic side view showing an example of FIG. 1A when in use;

FIG. 2A is a partially enlarged schematic side view showing an embodiment;

FIG. 2B is a schematic cross-sectional view showing enlarged a first expanded diameter portion of the core shaft in FIG. 2A;

FIG. 3A is a partially enlarged schematic side view showing an embodiment;

FIG. 3B is a partially enlarged schematic side view showing an embodiment;

FIG. 3C is a partially enlarged schematic side view showing an embodiment;

FIG. 4 is a partially enlarged schematic side view showing an embodiment;

FIG. 5 is a partially enlarged schematic side view showing an embodiment; and

FIG. 6 is a partially enlarged schematic side view showing an embodiment.

### DETAILED DESCRIPTION

The guide wire includes a core shaft having a first expanded diameter portion which expands, i.e., increases, in diameter toward a proximal end side and a first extended portion having a cylindrical shape or having a truncated cone shape that becomes thinner, e.g., a diameter thereof decreases, toward a distal end side. The first extended portion, which is adjacent to the first expanded diameter portion and extends along a longitudinal axis direction. A first coil body may be wound so as to cover at least a portion of the core shaft. A distal end fixing portion fixes a distal end of the core shaft and a distal end of the first coil body to each other. A gradient of a proximal end of the first expanded diameter portion may be greater than a gradient of a distal end of the first expanded diameter portion. An average gradient between a distal end of the first expanded diameter portion and a proximal end of the first expanded diameter portion may be greater than a gradient of the first extended portion. A gradient of the first expanded diameter portion may increase continuously toward a proximal end side. A proximal end of the first coil body may not be fixed to the first expanded diameter portion, but may be fixed to the first extended portion.

In the present disclosure, the “distal end side” is a direction along the longitudinal axis direction of the guide wire, and refers to the direction in which the distal end fixing portion is located with respect to the first coil body. The “proximal end side” is a direction along the longitudinal axis direction of the guide wire, and refers to the opposite direction to the distal end side. In addition, the “distal end” refers to the end portion of any member or part on the distal end side, and the “proximal end” refers to the end portion of any member or part on the proximal end side. Furthermore, the term “gradient” refers to the amount of increase in the radius of the core shaft per unit distance in the longitudinal axis direction. The “longitudinal axis” refers to the central axis of the core shaft, and the “longitudinal axis direction” refers to a direction along the longitudinal axis.

The “distal end portion” refers to a part which includes the distal end of any member or part, which extends from the distal end to a part in the middle toward the proximal end side of the member or the like. The “proximal end portion” refers to a portion which includes the proximal end of any member or part, which extends from the proximal end to a part in the middle toward the distal end side of the member or the like.

Some embodiments of the present disclosure will be described below with reference to the drawings, but the present disclosure is not limited only to the embodiments described in the drawings. Note that the dimensions of the guide wire illustrated in the drawings are dimensions shown for the purpose of facilitating an understanding of the implementation details, and do not necessarily correspond to the actual dimensions.

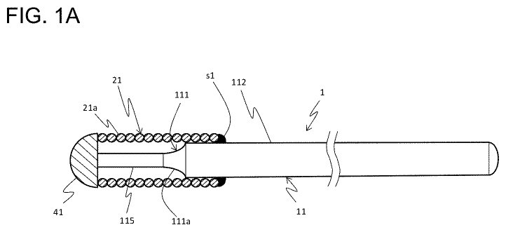

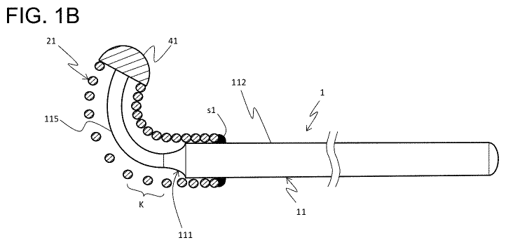

FIG. 1A is a schematic side view showing an embodiment. As illustrated in FIG. 1A, a guide wire 1 is schematically composed of a core shaft 11, a first coil body 21, and a distal end fixing portion 41.

The core shaft 11 is a shaft in which the distal end portion has a diameter which reduces stepwise toward the distal end side. For example, the core shaft 11 is configured to have a first expanded diameter portion 111, i.e., a variable diameter portion having a largest diameter at the proximal end side and a smallest diameter at the distal end side, a first extended portion 112 adjacent to the first expanded diameter portion 111 at the proximal end side and extending along the longitudinal axis direction towards the proximal end side, and a small diameter portion 115 adjacent to the first expanded diameter portion 111 at the distal end side and extending along the longitudinal axis direction towards the distal end side. A diameter of the first extended portion 112 may match the diameter of the first expanded diameter portion 111 at the proximal end side and a diameter of the small diameter portion 115 may match the diameter of the first expanded diameter portion 111 at the distal end side

The first expanded diameter portion 111 is a part of the core shaft 11 in which the diameter expands toward the proximal end side. The gradient of the proximal end of the first expanded diameter portion 111 may be greater than the gradient of the distal end of the first expanded diameter portion 111. The average gradient between the distal end of the first expanded diameter portion 111 and the proximal end of the first expanded diameter portion 111 may be greater than the gradient of the first extended portion 112. The gradient of the first expanded diameter portion 111 may increase continuously toward the proximal end side. Specifically, the first expanded diameter portion 111 may adopt, for example, a structure in which the gradient gradually changes along the longitudinal axis direction (a structure in which an outer peripheral surface 111a of the first expanded diameter portion 111 has a curved shape in a cross-section including the longitudinal axis) or a structure that uses a plurality of truncated cones having different gradients that are joined such that the end surfaces coincide with each other, and which are arranged such that the truncated cone having the smaller gradient is on the distal end side of the first expanded diameter portion, and the truncated cone having the larger gradient is on the proximal end side of the first expanded diameter portion; or structure that combines these structures.

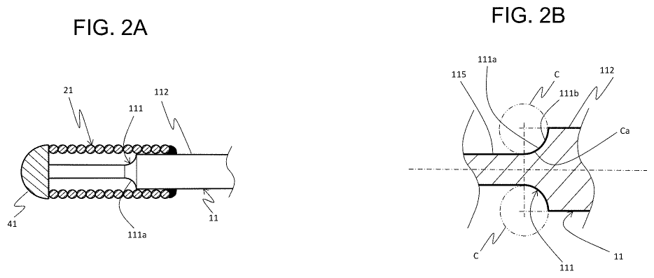

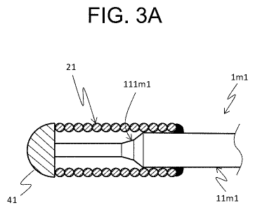

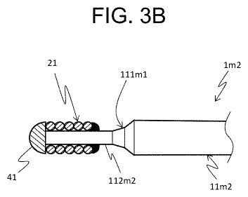

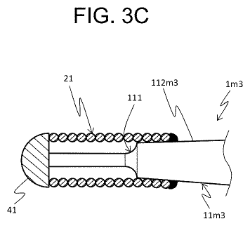

The first extended portion 112 is a part of the core shaft 11 having a cylindrical shape (see FIG. 2A) or a truncated cone shape that becomes thinner toward the distal end side (see FIG. 3C). The first extended portion 112 is adjacent to the first expanded diameter portion 111 and extends along the longitudinal axis direction from the first expanded diameter portion 111 to the proximal end side.

Here, the gradient of the first expanded diameter portion 111 described above may gradually change along the longitudinal axis direction. As a result, the rigidity of the core shaft 11 in the longitudinal axis direction can be smoothly changed along the longitudinal axis direction, and plastic deformation of the guide wire 1 at the first expanded diameter portion 111 may be preventable.

Furthermore the gradient of the distal end of the first expanded diameter portion 111 may be zero and the proximal end of the first expanded diameter portion 111 may be perpendicular to the longitudinal axis (the surface of the proximal end of the first expanded diameter portion 111 may be perpendicular to the longitudinal axis). An example of such a first expanded diameter portion 111 is a structure in which a contour line 111b of the outer peripheral surface 111a of the first expanded diameter portion 111 in a cross-section including the axis L is an arc Ca equivalent to one quarter of the circumference C (see FIG. 2A and FIG. 2B). In FIG. 2B, the double-dotted line represents the circumference C shown hypothetically. As a result, the rigidity of the core shaft 11 in the longitudinal axis direction can be smoothly and monotonically changed, and plastic deformation of the guide wire 1 at the first expanded diameter portion 111 can be effectively prevented.

In the guide wire 1, the core shaft 11 may be configured such that the gradient of the first expanded diameter portion 111 gradually changes along the longitudinal axis direction (the outer peripheral surface 111a is curved), and the cylindrical first extended portion 112 (constant outer diameter) extends toward the proximal end side to be continuous with the proximal end of the first expanded diameter portion 111. Furthermore, the distal end of the first expanded diameter portion 111 may be continuous with a cylindrical small diameter portion 115 (constant outer diameter) extending toward the distal end side.

In terms of the dimensions of the core shaft 11 in the longitudinal axis direction, the total length may be 1,800 to 3,000 mm, and the first expanded diameter portion 111 may be 1 mm to 3 mm. The outer diameter of the core shaft 11 may be 0.25 mm to 0.46 mm at the first extended portion 112.

The first coil body 21 is a helical (coiled) member wound to cover at least a portion of the core shaft 11. The first coil body 21 may adopt, for example, a structure in which a wire 21a is wound to form a coil shape (see FIG. 1A), or a structure in which a cylindrical member is subjected to slit processing or the like to form a coil shape. When the wire 21a is used for the first coil body 21, the wire 21a may be one or more solid wires, one or more twisted wires, and the like. Note that, a solid wire refers to a single wire, and a twisted wire refers to a bundled group of wires formed by twisting a plurality of single wires with each other in advance.

The diameter of the wire 21a (solid wire or twisted wire) constituting the first coil body 21 may be 0.01 to 0.10 mm.

In terms of the wire material constituting the first coil body 21, from the perspective of ensuring the flexibility of the guide wire 1 and imparting antithrombogenicity and biocompatibility, for example, stainless steel as SUS316; a superelastic alloy such as an Ni—Ti alloy; or a radiopaque metal such as platinum or tungsten may be adopted.

The proximal end of the first coil body 21 may not be fixed to the first expanded diameter portion 111, but may be fixed to the first extended portion 112 (joint part s1 in FIG. 1A). In terms of the method of fixing the proximal end of the first coil body 21 with the first extended portion 112, for example, a method that joins by means of a brazing method using a brazing material may be adopted. Examples of the brazing material include brazing metals such as an Sn—Pb alloy, a Pb—Ag alloy, an Sn—Ag alloy, an Au—Sn alloy, and the like.

The distal end fixing portion 41 is a part in which the distal end of the core shaft 11 and the distal end of the first coil body 21 may be integrally fixed. Specifically, for example, the distal end fixing portion 41 is formed so that the distal end portion forms a substantially hemispherical shape which is curved in a convex shape toward the distal end side. As a result, the resistance can be reduced when the guide wire 1 moves forward in a blood vessel, and the guide wire 1 can be smoothly inserted.

Examples of the method of forming the distal end fixing portion 41 that can be adopted include a method that forms the distal end fixing portion 41 by melting and molding a portion of the members constituting the core shaft 11 and/or the first coil body 21, and a method that forms the distal end fixing portion 41 by joining the core shaft 11 and the first coil body 21 using a brazing material, and then molding the brazing material. Examples of the brazing material include the same materials as those described above in the method of fixing the proximal end of the first coil body 21.

Next, the manner in which the guide wire 1 is used will be explained. First, the guide wire 1 is inserted from the distal end into a blood vessel, and the distal end portion is pushed forward by operating the guide wire 1 exposed outside the body. To improve operability and the like, the distal end portion of the guide wire 1 may be intentionally bent prior to insertion to the blood vessel, or a U-shaped curved portion may be formed by an unintentional bending force while proceeding through the blood vessel.

Here, although a proximal end portion K of the curved portion described above (see FIG. 1B) typically proceeds (moves) to the proximal end side as the guide wire 1 is pushed into the blood vessel. However, it is difficult for the proximal end portion K of the curved portion to proceed beyond the first expanded diameter portion 111 to the proximal end side, and the core shaft 11 does not easily become plastically bent at the first expanded diameter portion 111. Therefore, the guide wire 1 can be efficiently inserted up to the treatment site due to the excellent operability.

Then, after the distal end portion of the guide wire 1 reaches the treatment site, the proximal end of the guide wire 1 is inserted from the distal end of a medical device such as a catheter and into an inner cavity of the device. Once the guide wire 1 protrudes from the proximal end of the medical device, the medical device is pushed forward along the guide wire 1. Next, once the medical device reaches the treatment site, various treatments can be performed using the medical device. Then, after completion of the treatments, the series of procedures is completed by withdrawing the medical device and the guide wire 1 from the blood vessel.

Because the guide wire 1 has the configuration described above, it is possible to prevent the proximal end portion K of the U-shaped curved portion formed on the distal end portion of the guide wire 1 from proceeding to the proximal end side beyond the first expanded diameter portion 111, and plastic deformation of the core shaft 11 at the first expanded diameter portion 111 can be prevented. Therefore, the guide wire 1 can be smoothly operated in a blood vessel and efficiently inserted up to the treatment site. Here, because the proximal end of the first coil body 21 is not fixed to the first expanded diameter portion 111, the rigidity difference in the longitudinal axis direction can be made large before and after the first expanded diameter portion 111. This large rigidity difference may prevent the proximal end portion K of the curved portion from proceeding. In addition, the gradient of the first expanded diameter portion 111 may be greater than the gradient of the first extended portion 112 and the gradient of the proximal end of the first expanded diameter portion 111 may be greater than the gradient of the distal end of the first expanded diameter portion 111. Thus, the rate of change in the rigidity of the first expanded diameter portion 111 in the longitudinal axis direction can be made more gradual. This may prevent plastic deformation of the first expanded diameter portion 111.

In the above-mentioned embodiment, the guide wire 1 in which the gradient of the first expanded diameter portion 111 gradually changes along the longitudinal axis direction has been described. However, as shown in FIG. 3A, a guide wire 1m1 may include a core shaft 11m1 having a first expanded diameter portion 111m1, in which a plurality of truncated cones having different gradients are arranged along the longitudinal axis direction, with the end surfaces of the truncated cones coincide with each other, i.e., the diameters thereof match.

Furthermore, in the above-mentioned embodiment, the guide wire 1 in which the first extended portion 112 is arranged on the proximal end side of the first expanded diameter portion 111, and which extends toward the proximal end side in the longitudinal axis direction such that the distal end is continuous with the proximal end of the first expanded diameter portion 111 has been described. However, as shown in FIG. 3B, a guide wire 1m2 may include a core shaft 11m2, in which a first extended portion 112m2 is arranged on the distal end side of the first expanded diameter portion 111, and which extends toward the distal end side in the longitudinal axis direction such that the proximal end of the first extended portion 112m2 is continuous with the distal end of the first expanded diameter portion 111. In other words, the first extended portion 112m2 may serve as the small diameter portion 115. Further, while previous embodiments the first coil body 21 covers the first expanded portion 111 and is fixed to the core shaft adjacent to the proximal end of the first expanded portion, as shown in FIG. 3B, the first coil body 21 may not cover the first expanded portion 111 and may be fixed to the first extended portion 112m2 adjacent to the distal end of the first expanded portion 111.

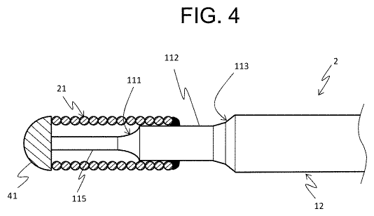

FIG. 4 is a partially enlarged schematic side view showing an embodiment. As illustrated in FIG. 4, a guide wire 2 is schematically configured by a core shaft 12, a first coil body 21, and a distal end fixing portion 41. The guide wire 2 differs from the guide wire 1 in that the core shaft 12 has a second expanded diameter portion 113. Since the configurations other than the configuration of the core shaft 12 are the same as those of the guide wire 1, the same parts are designated by the same reference numerals and detailed description thereof will not be repeated. Furthermore, the manner in which the guide wire 2 is used is the same as that of the guide wire 1, and detailed description thereof will not be repeated.

The core shaft 12 is a shaft in which the distal end portion has a diameter which reduces stepwise toward the distal end side. For example, the core shaft 12 has a first expanded diameter portion 111, a second expanded diameter portion 113, a first extended portion 112, and a small diameter portion 115.

The first expanded diameter portion 111 is a part of the core shaft 12 in which the diameter expands, i.e., increases, toward the proximal end side. The gradient of the proximal end of the first expanded diameter portion 111 may be greater than the gradient of the distal end of the first expanded diameter portion 111, the average gradient from the distal end to the proximal end of the first expanded diameter portion 111 may be greater than the gradient of the first extended portion 112, and the gradient of the first expanded diameter portion 111 may increase continuously toward the proximal end side.

The second expanded diameter portion 113 is a part of the core shaft 12 in which the diameter expands, i.e., increases, toward the proximal end side. For example, the second expanded diameter portion 113 may be arranged on the proximal end side of the first extended portion 112 described below, and can be provided extending may extend toward the proximal end side in the longitudinal axis direction such that the distal end of the second expanded diameter portion 113 is continuous with the proximal end of the first extended portion 112.

The gradient of the second expanded diameter portion 113 may be greater than the gradient of the first extended portion 112, and the gradient of the proximal end of the second expanded diameter portion 113 may be greater than the gradient of the distal end of the second expanded diameter portion 113. An example of such a second expanded diameter portion 113 is a structure that uses truncated cones having different gradients, which are arranged along the longitudinal axis direction such that the truncated cone having the smaller gradient is on the distal end side of the second expanded diameter portion 113 and the truncated cone having the larger gradient is on the proximal end side of the second expanded diameter portion 113, and the truncated cones are joined such that the end surfaces coincide with each other. As a result, the rigidity difference of the second expanded diameter portion 113 in the longitudinal axis direction compared to the first extended portion 112 may be increased. Therefore, the proximal end portion K of the curved portion can be prevented from proceeding in multiple stages due to the large rigidity difference of the second expanded diameter portion 113 in addition to the first expanded diameter portion 111. Furthermore, when the gradient of the proximal end of the second expanded diameter portion 113 is greater than the gradient of the distal end of the second expanded diameter portion 113, plastic deformation of the second expanded diameter portion 113 can also be prevented.

Furthermore, the gradient of the second expanded diameter portion 113 may be greater than the gradient of the first extended portion 112, and may be constant along the longitudinal axis direction. As a result, the rigidity difference of the second expanded diameter portion 113 in the longitudinal axis direction compared to the first extended portion 112 may be increased. Therefore, the proximal end portion K of the curved portion can be prevented from proceeding proximally in multiple stages due to the large rigidity difference of the second expanded diameter portion 113 in addition to the first expanded diameter portion 111.

The first extended portion 112 is a part of the core shaft 12 which is adjacent to the first expanded diameter portion 111. For example, the first extended portion 112 may have a cylindrical shape or a truncated cone shape that becomes thinner toward the distal end side, and extend along the longitudinal axis direction. The first extended portion 112 may be between the first expanded diameter portion 111 and the second expanded diameter portion 113.

In the guide wire 2, the core shaft 12 may be configured such that the gradient of the first expanded diameter portion 111 gradually changes along the longitudinal axis direction (the outer peripheral surface 111a is curved), and the cylindrical small diameter portion 115 (constant outer diameter) is provided on the distal end of the first expanded diameter portion 111 so as to extend toward the distal end side. The second expanded diameter portion 113 may use two truncated cones having different gradients arranged along the longitudinal axis direction such that the truncated cone having the smaller gradient is on the distal end side of the second expanded diameter portion 113 and the truncated cone having the larger gradient is on the proximal end side of the second expanded diameter portion 113, and the truncated cones may be joined such that the end surfaces coincide with each other. The first extended portion 112 may have a cylindrical shape extending along the longitudinal axis direction, and may be between the first expanded diameter portion 111 and the second expanded diameter portion 113 such that the distal end of the first extended portion 112 is adjacent to the proximal end of the first expanded diameter portion 111 and the proximal end of the first extended portion 112 is adjacent to the distal end of the second expanded diameter portion 113.

As a result of the guide wire 2 having the configuration described above, it is possible to prevent the proximal end portion of the U-shaped curved portion formed on the distal end portion of the guide wire 2 from proceeding beyond the first expanded diameter portion 111 to the proximal end side, and plastic deformation of the core shaft 12 at the first expanded diameter portion 111 can be prevented. Furthermore, because the guide wire 2 is provided with the second expanded diameter portion 113, even if the proximal end portion of the curved portion proceeds beyond the first expanded diameter portion 111 to the proximal end side, the position of the proximal end portion and the degree of curvature of the U-shaped curved portion can be adjusted in multiple stages according to a pushing force of the guide wire 2.

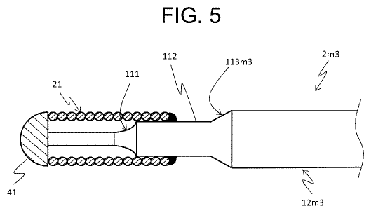

In the guide wire 2 described above, although the guide wire 2 provided with the core shaft 12 in which the gradient of the proximal end of the second expanded diameter portion 113 is greater than the gradient of the distal end of the second expanded diameter portion 113 has been described, as shown in FIG. 5, a guide wire 2m3 which includes a core shaft 12m3 having a second expanded diameter portion 113m3 may have a constant gradient of the second expanded diameter portion 113m3.

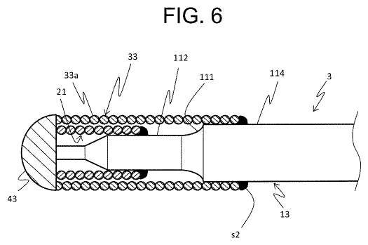

FIG. 6 is a partially enlarged schematic side view showing an embodiment. As illustrated in FIG. 6, a guide wire 3 is schematically composed of a core shaft 13, a first coil body 21, a second coil body 33, and a distal end fixing portion 43. The guide wire 3 differs from the guide wire 1 in that the core shaft 13, the second coil body 33, and the distal end fixing portion 43 are provided. Since the configurations other than the configurations of the core shaft 13, the second coil body 33, and the distal end fixing portion 43 are the same as those of the guide wire 1, the same parts are designated by the same reference numerals and detailed description thereof will not be repeated. Furthermore, the manner in which the guide wire 3 is used is the same as that of the guide wire 1, and detailed description thereof will not be repeated.

The core shaft 13 is a shaft in which the distal end portion has a diameter which reduces stepwise toward the distal end side. The core shaft 13 includes a first expanded diameter portion 111, a first extended portion 112, and a second extended portion 114.

The first expanded diameter portion 111 is a part of the core shaft 13 in which the diameter expands toward the proximal end side. The gradient of the proximal end of the first expanded diameter portion 111 is greater than the gradient of the distal end of the first expanded diameter portion 111, the average gradient from the distal end to the proximal end of the first expanded diameter portion 111 is greater than the gradient of the first extended portion 112, and the gradient of the first expanded diameter portion 111 increases continuously toward the proximal end side.

The first extended portion 112 is a part of the core shaft 13 arranged on the distal end side of the first expanded diameter portion 111. The first extended portion 112 can be configured to have a cylindrical shape or a truncated cone shape that becomes thinner toward the distal end side, be adjacent to the first expanded diameter portion 111 and to extend along the longitudinal axis direction.

The second extended portion 114 is a part of the core shaft 13 arranged on the proximal end side of the first expanded diameter portion 111. The second extended portion 114 may be configured to have a cylindrical shape or a truncated cone shape that becomes thinner toward a distal end side, and extend along the longitudinal axis direction.

In the guide wire 3, the gradient of the first expanded diameter portion 111 gradually changes along the longitudinal axis direction, and the outer peripheral surface 111a of the first expanded diameter portion 111 is curved. The first extended portion 112 is cylindrical (constant outer diameter) on the distal end side of the first expanded diameter portion 111, and extends toward the distal end side in the longitudinal axis direction such that the proximal end of the first extended portion 112 is continuous with the distal end of the first expanded diameter portion 111. The second extended portion 114 is cylindrical (constant outer diameter) on the proximal end side of the first expanded diameter portion 111, and extends toward the proximal end side in the longitudinal axis direction such that the distal end of the second extended portion 114 is continuous with the proximal end of the first expanded diameter portion 111.

The second coil body 33 is a helical (coiled) member wound to cover the first coil body 21. The second coil body 33 may adopt, for example, a structure in which a wire 33a is wound to form a coil shape (see FIG. 6), or a structure in which a cylindrical member is subjected to slit processing or the like to form a coil shape. When the wire 33a is used as the second coil body 33, it is possible to use, for example, a wire equivalent to the wire 21a of the first coil body 21 described in the guide wire 1 as the wire 33a.

The proximal end of the second coil body 33 may not be fixed to the first expanded diameter portion 111, but may be fixed to the second extended portion 114 (joint part s2 in FIG. 6). On the other hand, the distal end of the second coil body 33 may, for example, be integrally fixed to the distal end fixing portion 43 together with the distal end of the core shaft 13 and the distal end of the first coil body 21.

As described above, because the guide wire 3 has the above configuration, even if the core shaft 13 breaks apart at the first expanded diameter portion 111, the distal end side and the proximal end side of the broken core shaft 13 are connected via the second coil body 33, and the distal end side and the proximal end side of the core shaft 13 can be prevented from separating.

Note that the present invention is not limited to the configurations of the embodiments described above, but is stipulated by claims, and the present invention is intended to include all modifications within the meaning and scope equivalent to those in claims.

1. A guide wire comprising:
a core shaft having a first expanded diameter portion which increases in diameter toward a proximal end side and a first extended portion, adjacent to the first expanded diameter portion and that extends along a longitudinal axis direction;
a first coil body wound to cover at least a portion of the core shaft; and
a distal end fixing portion at which a distal end of the core shaft and a distal end of the first coil body are fixed to each other; wherein
a gradient of a proximal end of the first expanded diameter portion is greater than a gradient of a distal end of the first expanded diameter portion,
an average gradient between a distal end of the first expanded diameter portion and a proximal end of the first expanded diameter portion is greater than a gradient of the first extended portion,
a gradient of the first expanded diameter portion increases continuously toward a proximal end side, and
a proximal end of the first coil body is not fixed to the first expanded diameter portion, but is fixed to the first extended portion.

2. The guide wire according to claim 1, wherein
the gradient of the first expanded diameter portion gradually changes along a longitudinal axis direction.

3. The guide wire according to claim 2, wherein
the gradient at the distal end of the first expanded diameter portion is zero, and the proximal end of the first expanded diameter portion stands perpendicular to a longitudinal axis of the core shaft.

4. The guide wire according to claim 1, wherein
the core shaft includes a second expanded diameter portion which expands in diameter toward a proximal end side, and
the first extended portion is between the first expanded diameter portion and the second expanded diameter portion.

5. The guide wire according to claim 4, wherein
a gradient of the second expanded diameter portion is greater than the gradient of the first extended portion, and
a gradient of the proximal end of the second expanded diameter portion is greater than a gradient of a distal end of the second expanded diameter portion.

6. The guide wire according to claim 4, wherein
a gradient of the second expanded diameter portion is greater than a gradient of the first extended portion, and is constant along a longitudinal axis direction.

7. The guide wire according to claim 1, comprising:
a second coil body wound to cover the first coil body, wherein
the core shaft includes the first extended portion adjacent to the distal end side of the first expanded diameter portion, and a second extended portion adjacent to the proximal end side of the first expanded diameter portion and extends along a longitudinal axis direction, and
a proximal end of the second coil body is not fixed to the first expanded diameter portion, but is fixed to the second extended portion.

8. The guide wire according to claim 7, wherein the first extended portion has a cylindrical shape or has a truncated cone shape that decreases toward a distal end side.

9. The guide wire according to claim 7, wherein the second extended portion has a cylindrical shape or has a truncated cone shape that decreases toward a distal end side.

10. The guide wire according to claim 1, wherein the first extended portion has a cylindrical shape or has a truncated cone shape that decreases toward a distal end side.

11. The guide wire according to claim 1, wherein the first extended portion is adjacent to the distal end side of the first expanded diameter portion.

12. The guide wire according to claim 1, wherein the first extended portion is adjacent to the proximal end side of the first expanded diameter portion.

13. The guide wire according to claim 12, wherein the first coil body covers the first expanded diameter portion.

14. The guide wire according to claim 1, wherein the gradient of the distal end of the first expanded diameter portion is zero.
